(19) United States
(12) Patent Application Publication
(10) Pub. No.: US 2025/0223604 A1
(43) Pub. Date: Jul. 10, 2025

(54) WHEAT VOLTAGE-DEPENDENT ANION CHANNEL GENE TaVDAC1 AND USE THEREOF

(71) Applicant: Northwest A&F University, Yangling (CN)

(72) Inventors: Qinhu WANG, Yangling (CN); Xiaolin TIAN, Yangling (CN); Huiquan LIU, Yangling (CN)

(21) Appl. No.: 19/007,988

(22) Filed: Jan. 2, 2025

(30) Foreign Application Priority Data
Jan. 4, 2024 (CN) 202410008092.1

(51) Int. Cl. C12N 15/82; C12N 15/11

(52) U.S. Cl. CPC C12N 15/8218; C12N 15/11; C12N 15/8282

(57) ABSTRACT

The present disclosure belongs to the field of genetic engineering technology and relates to a voltage-dependent anion channel gene TaVDAC1 of wheat and use thereof. The wheat voltage-dependent anion channel gene TaVDAC1 negatively regulates wheat resistance to Fusarium head blight (FHB). When the gene TaVDAC1 is overexpressed, the resistance of wheat to FHB decreases; conversely, when the gene TaVDAC1 is silenced or edited, the resistance of wheat to FHB significantly increases. The wheat voltage-dependent anion channel gene TaVDAC1 may be used for the breeding and development of crop variety or germplasm resources with resistance to FHB, thereby enhancing wheat disease resistance and addressing the technical issues of wheat susceptibility to FHB and the reliance on chemical control methods for its management.

### CROSS-REFERENCE TO RELATED APPLICATION

This patent application claims the benefit and priority to the Chinese Patent Application No. 202410008092.1 filed with the China National Intellectual Property Administration on Jan. 4, 2024, the disclosure of which is incorporated by reference herein in its entirety as part of the present application.

### REFERENCE TO SEQUENCE LISTING

A computer readable XML file entitled “GWP20241207902_seqlist”, which was created on Dec. 13, 2024, with a file size of about 9,644 bytes, contains the sequence listing for this application, has been filed with this application, and is hereby incorporated by reference in its entirety.

### TECHNICAL FIELD

The present disclosure belongs to the field of bioengineering technology and relates to wheat voltage-dependent anion channel gene TaVDAC1 and use thereof.

### BACKGROUND

The yield and quality of wheat are of significant importance for food security. Fusarium head blight (FHB) caused by Fusarium graminearum species complex is one of the three major fungal diseases affecting wheat. With the ongoing global warming and changes in cultivation practices, the frequency and severity of FHB outbreaks have significantly increased in recent years. This disease directly damages the wheat spike, leading to shriveled grains and severely impacted wheat yield. Additionally, the mycotoxin deoxynivalenol (DON), which resided in the grains, is harmful to both human and livestock.

Currently, chemical control remains the primary approach for managing FHB and reducing mycotoxin contamination. However, with the frequent use of chemical fungicides, the resistance risk of field strains, as well as the challenge of FHB management, have been increasing. Breeding of resistant crops is one of the most economically effective approaches to combat FHB in wheat. However, the genetic resources for FHB resistance are scarce, and their mechanisms of resistance are complex. In particular, the cloning of these resistant genes is time-consuming. The lack of major effective resistance genes hinders the conventional molecular breeding process and makes it difficult to develop resistant varieties. Therefore, it is imperative to explore innovative approaches for the development of FHB-resistant wheat varieties, to enhance the effectiveness of FHB management.

### SUMMARY

Developing resistant varieties is the most direct, effective, and economical approach for controlling FHB in wheat. Embodiments of the present disclosure aims to further explore functional genes that regulate wheat resistance to FHB, and employ effective strategies to expand the use of these functional genes in enhancing wheat resistance. This will uncover key regulators of FHB resistance and provide new technical approaches for molecular-assisted breeding and the development of resistant plants.

In line with these objectives, the present disclosure provides a wheat voltage-dependent anion channel gene TaVDAC1 and use thereof to meet the needs in this field, overcoming the technical deficiencies associated with chemical and biological control of FHB in wheat.

In one aspect, the present disclosure relates to a wheat voltage-dependent anion channel gene TaVDAC1, where the wheat voltage-dependent anion channel gene TaVDAC1 negatively regulates wheat resistance against FHB and has the nucleotide sequence of SEQ ID NO: 1.

In some embodiments, the gene TaVDAC1 encodes a wheat voltage-dependent anion channel protein having the amino acid sequence of SEQ ID NO: 2.

In some embodiments, the gene TaVDAC1 provided by the present disclosure has a fragment having the nucleotide sequence of SEQ ID NO: 3 for silencing.

In some embodiments, the present disclosure further relates to a wheat voltage-dependent anion channel protein TaVDAC1, where the protein TaVDAC1 is encoded by the gene TaVDAC1 and has the amino acid sequence of SEQ ID NO: 2.

In another aspect, the present disclosure relates to a vector for gene silencing or gene editing, including elements for silencing or editing the gene TaVDAC1.

Experimental validation demonstrates that silencing or editing the gene TaVDAC1 can enhance wheat resistance to FHB. Therefore, the present disclosure further claims the use of the gene TaVDAC1, the protein TaVDAC1, or the vector for gene silencing or gene editing in developing FHB-resistant wheat variety or germplasm.

In some embodiments, in the use provided by the present disclosure, silencing the gene TaVDAC1 enhances wheat resistance to FHB.

In some embodiments, in the use provided by the present disclosure, the gene editing results in deletion or inactivation of the gene TaVDAC1, which enhances wheat resistance to FHB.

Compared to the prior art, embodiments provided by the present disclosure have the following beneficial effects or advantages.

The present disclosure provides a wheat voltage-dependent anion channel gene, TaVDAC1, which plays a negative regulatory role in wheat resistance to FHB. This disclosure involves the wheat voltage-dependent anion channel gene TaVDAC1, the amino acids encoded by the gene TaVDAC1, as well as a sequence for silencing and editing the gene TaVDAC1 in wheat.

When the gene TaVDAC1 is overexpressed, the resistance of wheat to F. graminearum decreases, leading to enhanced susceptibility. By employing RNA interference technology, the expression level of the voltage-dependent anion channel gene TaVDAC1 is reduced through the silencing of the specific gene fragment (SEQ ID NO: 3), resulting in increased FHB resistance of wheat to F. graminearum. Additionally, using Clustered Regularly Interspaced Short Palindromic Repeats associated with Cas9 nuclease (CRISPR-Cas9) gene editing technology, editing the gene TaVDAC1 also enhances wheat resistance to F. graminearum.

The gene TaVDAC1 may be utilized in the breeding and generation of FHB-resistant wheat by reducing crop susceptibility to this disease, thereby reducing the reliance on chemical control for FHB.

### BRIEF DESCRIPTION OF THE DRAWINGS

To clarify the embodiments of the present disclosure or the prior art, the following is a brief introduction to the accompanying drawings used in the description of the examples or prior art. It is evident that the drawings described below are merely some embodiments of the present disclosure. Those skilled in the art can derive other embodiments based on these without requiring creative effort.

FIG. 1 illustrates the gel electrophoresis results of the wheat voltage-dependent anion channel gene TaVDAC1. The left lane in FIG. 1 shows the Marker bands, while the right lane displays the band of the target gene.

FIGS. 2A-2B illustrate the relative expression levels of the wheat voltage-dependent anion channel gene TaVDAC1 in the overexpression and silencing lines. In FIG. 2, “Fielder” represents the wild-type wheat; “RNAi” represents the transgenic line with silenced TaVDAC1; and “OE” represents the transgenic line with TaVDAC1 overexpression.

FIG. 3 illustrates the infection assay of the transgenic lines with silenced and overexpressed wheat voltage-dependent anion channel gene TaVDAC1. In FIG. 3, “Fielder” represents the wild-type wheat; “RNAi” represents the transgenic line with silenced TaVDAC1; and “OE” represents the transgenic line with TaVDAC1 overexpression.

FIG. 4 illustrates the infection assay of the wheat voltage-dependent anion channel gene (TaVDAC1)-edited lines. In FIG. 4, “Fielder” represents the wild-type wheat; “CR” represents the TaVDAC1 gene-edited lines generated with CRISPR-Cas9 technology.

### DETAILED DESCRIPTION OF THE EMBODIMENTS

Next, the embodiments of the present disclosure will be described in conjunction with the examples, but the disclosure is not limited to the following examples.

The experimental methods and detection methods described in the following examples are conventional methods unless otherwise specified; the reagents and materials mentioned can be purchased from the market unless otherwise indicated.

### Example 1

This example describes the isolation and cloning of the wheat voltage-dependent anion channel gene TaVDAC1. Pathogen effectors often promote infection by targeting key regulators of the host defense. During the screening of F. graminearum key effector targets in wheat, a wheat voltage-dependent anion channel gene encoding TaVDAC1 protein was identified.

The voltage-dependent anion channel gene TaVDAC1 was cloned from the wheat variety Fielder. The nucleotide sequence of the wheat voltage-dependent anion channel gene TaVDAC1 is set forth in SEQ ID NO: 1. The amino acid sequence of the protein encoded by the wheat voltage-dependent anion channel gene TaVDAC1 is set forth in SEQ ID NO: 2.

The spikes of the normal soil-cultivated wheat variety Fielder were inoculated with F. graminearum during the flowering stage. After three days, the lemma samples were collected, and the total RNAs were extracted and reverse-transcribed into cDNA.

The specific primers for the wheat voltage-dependent anion channel gene TaVDAC1, F1 (5′-ATGGGCGGCCCAGGCCTC-3′, SEQ ID NO: 4) and R1 (5′-TCAAGGCTTGAGAGCAA-3′, SEQ ID NO: 5), were used for PCR amplification, resulting in the amplification of the full-length coding sequence of the wheat voltage-dependent anion channel gene TaVDAC1.

The PCR reaction program was as follows: pre-denaturation at 95° C. for 5 minutes; denaturation at 95° C. for 30 seconds, annealing at 58.5° C. for 40 seconds, and extension at 72° C. for 1 minute for 35 cycles; a final extension at 72° C. for 10 minutes. The samples were stored at 4° C. The gel electrophoresis analysis indicated that the band size of PCR product is consistent with expectations, as shown in FIG. 1.

DNA sequencing further confirmed the cloned wheat voltage-dependent anion channel gene TaVDAC1 (as shown in SEQ ID NO: 1), with a full length of 828 bp, encoding 275 amino acids (as shown in SEQ ID NO: 2).

### Example 2

This example describes the cultivation of transgenic wheat overexpressing the voltage-dependent anion channel gene TaVDAC1 and the evaluation of its resistance against FHB.

The full-length wheat voltage-dependent anion channel gene TaVDAC1 was constructed into an overexpression vector using the Gateway method, resulting in the TaVDAC1-OE recombinant plasmid. After sequencing validation, the correct TaVDAC1-OE recombinant plasmid was transformed into Agrobacterium. Using the Agrobacterium infection method, the overexpression vector of the wheat voltage-dependent anion channel gene TaVDAC1 was introduced into the callus tissue of the wheat variety Fielder.

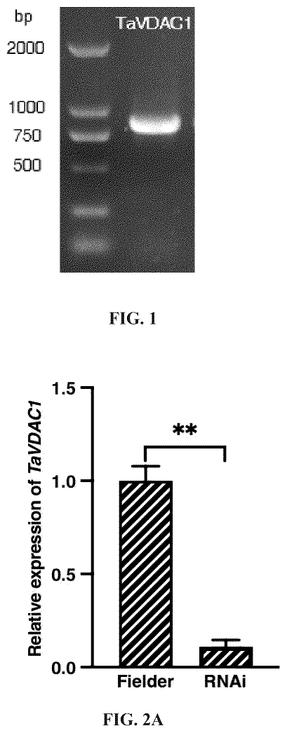

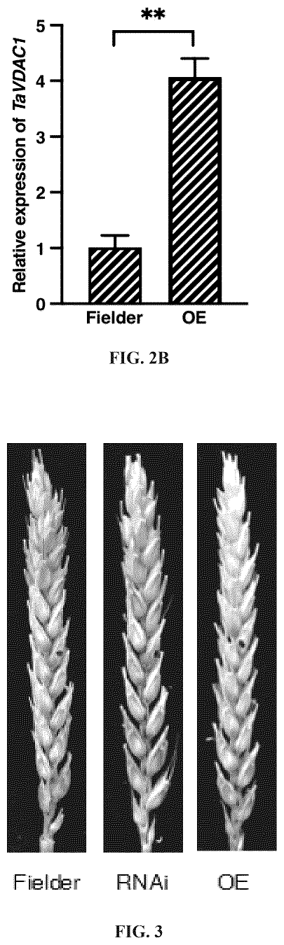

The T1 transgenic plants overexpressing the wheat voltage-dependent anion channel gene TaVDAC1 were selected for FHB resistance assessment. The OE plants, as well as the wild-type Fielder, were cultivated under a light/dark cycle of 16 hours/8 hours in the greenhouse. The quantitative reverse-transcription PCR (qRT-PCR) analysis showed that the expression level of TaVDAC1 in the OE plants was significantly higher compared to that in the wild-type (as shown in FIG. 2B).

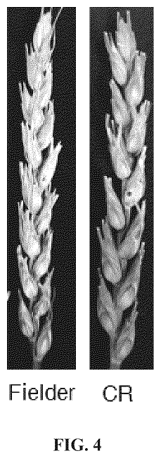

At the flowering stage, a suspension of F. graminearum conidial spores at a concentration of 2×10⁵ spores/mL was drop-inoculated into the middle part of the spike. After inoculation, the spikes were kept moist in a transparent plastic bag for 3 days, and the disease symptoms on the spikes were observed 11 days post-inoculation. As shown in FIG. 3, when the wheat voltage-dependent anion channel gene TaVDAC1 was overexpressed, the number of diseased spikelets was significantly higher compared to that in the control group (Fielder). Therefore, overexpression of TaVDAC1 gene promotes wheat susceptibly to FHB.

### Example 3

This example describes the cultivation of transgenic wheat with silenced voltage-dependent anion channel gene TaVDAC1 and its resistance evaluation against FHB.

Through sequence alignment and specificity analysis, a fragment targeting TaVDAC1 (the nucleotide sequence is set forth in SEQ ID NO: 3) was selected to construct the TaVDAC1-RNAi vector. The recombinant plasmid was transformed into Agrobacterium, and the TaVDAC1-RNAi vector was introduced into the wheat variety Fielder using Agrobacterium-mediated transformation to generate the transgenic plants.

The T1 generation of transgenic lines with silenced TaVDAC1 was selected for evaluation of resistance to FHB. The qRT-PCR results indicated that the expression level of TaVDAC1 in the RNAi plants was significantly reduced compared to the control group (Fielder), suggesting the effective silencing of the gene (as shown in FIG. 2A).

For the inoculation experiment, a suspension of F. graminearum conidial spores was drop-inoculated into the middle part of the spike. After 11 days of infection, the number of diseased spikelets in the RNAi lines was significantly lower compared to that in the wild-type (as shown in FIG. 3). The results indicate that silencing the wheat voltage-dependent anion channel gene TaVDAC1 enhances wheat resistance to FHB. This example confirms that the wheat voltage-dependent anion channel gene TaVDAC1 negatively regulates wheat resistance to FHB.

### Example 4

This example describes the cultivation of wheat edited for the voltage-dependent anion channel gene TaVDAC1 and its resistance evaluation against FHB.

Through the analysis of TaVDAC1 gene sequence, two guide RNAs (gRNAs) targeting the exon regions of wheat voltage-dependent anion channel gene TaVDAC1 were selected (gRNA1: 5′-GCTGACGATCTTGCAGCACC-3′, SEQ ID NO: 6; gRNA2: 5′-GGAGAGGTTGACTACAGGAT-3′, SEQ ID NO: 7). Using CRISPR-Cas9 gene editing technology, a gene editing vector for the wheat voltage-dependent anion channel gene TaVDAC1 was constructed. The recombinant plasmid was transformed into Agrobacterium, and the gene editing vector for TaVDAC1 was introduced into the wheat variety Fielder through Agrobacterium-mediated transformation, resulting in T0 generation plants.

The T1 generation plants, in which the gene TaVDAC1 had been edited across the wheat A, B, and D chromosomes, were selected for analysis of resistance to FHB. Fourteen days after inoculation with F. graminearum, the number of diseased spikelets in the edited mutant plants (CR) was significantly lower compared to that in the wild-type plants (as shown in FIG. 4). These data confirm that the deletion or the inactivation of the wheat voltage-dependent anion channel gene TaVDAC1 can improve wheat resistance to FHB.

In summary, the present disclosure successfully cloned the full-length of the wheat voltage-dependent anion channel gene TaVDAC1 from the cDNA of the wheat spike infected by F. graminearum (the nucleotide sequence is set forth in SEQ ID NO: 1). Through RNA interference technology and gene editing technology, it has been demonstrated that inhibiting or inactivating the wheat voltage-dependent anion channel gene TaVDAC1 increases wheat resistance to FHB.

As described above, the basic principles, main features, and advantages of the present disclosure have been well articulated. The above examples and descriptions are merely illustrations of preferred embodiments of the invention. The disclosure is not limited to the aforementioned examples, and various modifications and improvements made by those skilled in the art to the embodiments of the present disclosure without departing from the spirit and scope of the disclosure should fall within the protection scope defined by the present disclosure.

What is claimed is:

1. A vector for gene silencing or editing, comprising a specific fragment having the nucleotide sequence of SEQ ID NO: 3 or guide RNAs with the nucleotide sequence of SEQ ID NO: 6 and SEQ ID NO: 7, for silencing or editing the gene TaVDAC1.

2. A method for developing a Fusarium head blight (FHB)-resistant wheat variety or germplasm, comprising silencing or editing a wheat voltage-dependent anion channel gene TaVDAC1, wherein the wheat voltage-dependent anion channel gene TaVDAC1 negatively regulates the defense response of wheat against FHB and has the nucleotide sequence of SEQ ID NO: 1.

3. The method of claim 2, wherein silencing the gene TaVDAC1 or a specific fragment of the gene TaVDAC1 enhances wheat resistance to FHB.

4. The method of claim 2, wherein gene editing that results in deletion or inactivation of the gene TaVDAC1 enhances wheat resistance to FHB.

5. The method of claim 2, wherein the gene TaVDAC1 encodes a wheat voltage-dependent anion channel protein having the amino acid sequence of SEQ ID NO: 2.

6. The method of claim 2, wherein the gene TaVDAC1 has a specific fragment having the nucleotide sequence of SEQ ID NO: 3.

7. A method for developing a Fusarium head blight (FHB)-resistant wheat variety or germplasm, comprising abolishing an expression or activity of a wheat voltage-dependent anion channel protein TaVDAC1, wherein the protein TaVDAC1 is encoded by the gene TaVDAC1 defined in claim 2 and has the amino acid sequence of SEQ ID NO: 2.

8. A method for developing a Fusarium head blight (FHB)-resistant wheat variety or germplasm, comprising contacting the vector for gene silencing or editing of claim 1.


SEQUENCE LISTING (7 sequences)

SEQ ID NO: 1 - DNA, 828 bp, synthetic construct
  source 1..828: mol_type = other DNA; note = gene TaVDAC1
atgggcggcccaggcctctactccgggatcggcaagaaggccaaggatctgctatacagggactaccagaccgaccacaagttcaccctcaccacctacaccgccaatggcgccgcaattactgctaccagtacaaagaaggctgatcttatacttggtgagatccaatcacagatcaagaacaaaaacatcactgtagatgtgaaggcaaactcagcatcgaatgtcattactacaattaccgctgacgatcttgcagcaccagggctgaagaccatcttgagctttgctgttcctgatcagaaatctggaaaggttgagctccagtacttgcatgattatgctggtattaatgcaagcattggcttgactgccaatcctgtagtcaacctctccggtgcgttcggaactagtgctctagctgttggtgctgatgtgtcgcttgatacagccaccaagaatttcaccaaatacaatgctgcacttagctacactaaccaggatcttattgcatccctgaacctgaacaacaagggagacagcctgaccgcatcctattaccacattgtggagaagtccggcacagctgtcggggcggagctgacccacagcttctcgagcaacgagaacagcctcacctttggcacccagcataccctggacccgctcacccttgtgaaggcccggatcaacaactctggcaaggccagcgcgctgatccagcacgagttcatgccaaagtcgttgtgcaccatctcggcggaggtggacaccaaggccatcgagaagagctcgaaggtcggcattgctattgctctcaagccttga

SEQ ID NO: 2 - protein, 275 aa, synthetic construct
  source 1..275: note = protein TaVDAC1
MGGPGLYSGIGKKAKDLLYRDYQTDHKFTLTTYTANGAAITATSTKKADLILGEIQSQIKNKNITVDVKANSASNVITTITADDLAAPGLKTILSFAVPDQKSGKVELQYLHDYAGINASIGLTANPVVNLSGAFGTSALAVGADVSLDTATKNFTKYNAALSYTNQDLIASLNLNNKGDSLTASYYHIVEKSGTAVGAELTHSFSSNENSLTFGTQHTLDPLTLVKARINNSGKASALIQHEFMPKSLCTISAEVDTKAIEKSSKVGIAIALKP

SEQ ID NO: 3 - DNA, 289 bp, synthetic construct
  source 1..289: mol_type = other DNA; note = Specific fragment of gene TaVDAC1
ctacaccgccaatggcgccgcaattactgctaccagtacaaagaaggctgatcttatacttggtgagatccaatcacagatcaagaacaaaaacatcactgtagatgtgaaggcaaactcagcatcgaatgtcattactacaattaccgctgacgatcttgcagcaccagggctgaagaccatcttgagctttgctgttcctgatcagaaatctggaaaggttgagctccagtacttgcatgattatgctggtattaatgcaagcattggcttgactgccaatcctgta

SEQ ID NO: 4 - DNA, 18 bp, synthetic construct
  source 1..18: mol_type = other DNA; note = Primer F1
atgggcggcccaggcctc

SEQ ID NO: 5 - DNA, 17 bp, synthetic construct
  source 1..17: mol_type = other DNA; note = Primer R1
tcaaggcttgagagcaa

SEQ ID NO: 6 - DNA, 20 bp, synthetic construct
  source 1..20: mol_type = other DNA; note = gRNA1
gctgacgatcttgcagcacc

SEQ ID NO: 7 - DNA, 20 bp, synthetic construct
  source 1..20: mol_type = other DNA; note = gRNA2
ggagaggttgactacaggat